(19) United States
(12) Patent Application Publication
(10) Pub. No.: US 2016/0025265 A1
(43) Pub. Date: Jan. 28, 2016

(54) ACTIVE MONITORING OF HEAVY WEIGHT LUBRICATION OIL

(71) Applicant: Canrig Drilling Technology Ltd., Houston, TX (US)

(72) Inventors: Boone Elbert SMITH, Pinehurst, TX (US); Jose Abelardo SANCHEZ, Katy, TX (US)

(21) Appl. No.: 14/809,502

(22) Filed: Jul. 27, 2015

Related U.S. Application Data
(60) Provisional application No. 62/029,873, filed on Jul. 28, 2014.

(51) Int. Cl. F16N 29/04; G01N 33/28; F01M 11/10

(52) U.S. Cl. CPC F16N 29/04; F01M 11/10; G01N 33/2888

(57) ABSTRACT

A system for wellbore operations can be adapted to generate an alarm signal upon ingress of a contaminant into a heavy weight lubrication oil of an equipment prior to operational failure of the equipment. In an embodiment, the system can include a sensor. In an embodiment, the sensor can be adapted to sense a viscosity of the heavy weight lubrication oil, a density of the heavy weight lubrication oil, a dielectric of the heavy weight lubrication oil, a temperature of the heavy weight lubrication oil, or a combination thereof.

### CROSS-REFERENCE TO RELATED APPLICATION(S)

This application claims priority under 35 U.S.C. §119(e) to U.S. Patent Application No. 62/029,873, entitled “ACTIVE MONITORING OF HEAVY WEIGHT LUBRICATION OIL,” by Boone E. Smith and Jose A. Sanchez, filed Jul. 28, 2014, which is assigned to the current assignee hereof and incorporated herein by reference in its entirety.

### FIELD OF THE DISCLOSURE

The present invention relates generally to wellbore drilling operations, and more particularly to active monitoring of a heavy weight lubrication oil in an equipment on an oil rig and real-time relay of a condition of the heavy weight lubrication oil.

### RELATED ART

Drilling subterranean wells for oil and gas is expensive and time consuming. Formations containing oil and gas are typically located thousands of feet below the earth's surface. To access the oil and gas, thousands of feet of rock and other geological formations must be removed. To ensure a cost-effective drilling operation, equipment utilized in wellbore drilling operations must be capable of repeated, reliable operation, even when subjected to extreme environmental conditions. Repair or replacement of failed equipment can shut down an operation, rendering the drilling operation economically unsustainable.

Several of the equipment on a drill rig utilize heavy weight lubrication oil, e.g., SAE 90, SAE 140, or even SAE 250, within gearboxes and transmissions. Ingress of contaminant(s), e.g., mud, sand, metal particles, water, and other non-oil fluids, into the oil can mitigate the bearing properties thereof, increasing frictional resistance and accelerating wear and ultimate failure of the equipment.

The industry continues to demand improvements in subterranean drilling operations.

### BRIEF DESCRIPTION OF THE DRAWINGS

Embodiments are illustrated by way of example and are not limited in the accompanying figures.

FIG. 1 includes a schematic view of a drilling rig.

FIG. 2 includes a partially cut away top perspective view of a draw works including sensors in accordance with an embodiment.

FIG. 3 includes a simplified schematic of an active sensing and continuous (real time) information relaying operation adapted to sense and actively relay a condition of a heavy weight lubrication oil.

Skilled artisans appreciate that elements in the figures are illustrated for simplicity and clarity and have not necessarily been drawn to scale. For example, the dimensions of some of the elements in the figures may be exaggerated relative to other elements to help to improve understanding of embodiments of the invention.

### DETAILED DESCRIPTION

The following description in combination with the figures is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other embodiments can be used based on the teachings as disclosed in this application.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item may be used in place of a single item. Similarly, where more than one item is described herein, a single item may be substituted for that more than one item.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the drilling arts.

The concepts are better understood in view of the embodiments described below that illustrate and do not limit the scope of the present invention. The following description is directed to a system for wellbore operations adapted to actively monitor a condition of a heavy weight lubrication oil in an equipment on a drill rig and continuously (in real-time) relay the condition to a user. Moreover, the description is directed to a system adapted to sense ingress of a contaminant into a heavy weight lubrication oil of an equipment and generate an alarm signal in response to ingress of a contaminant.

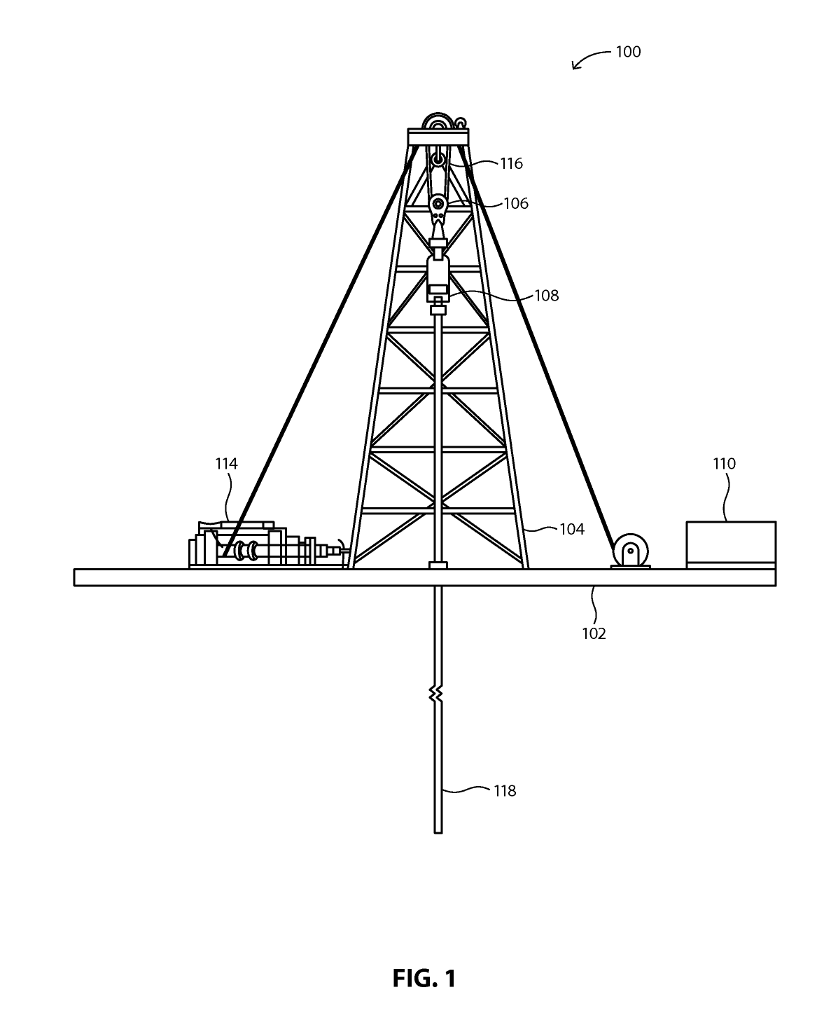

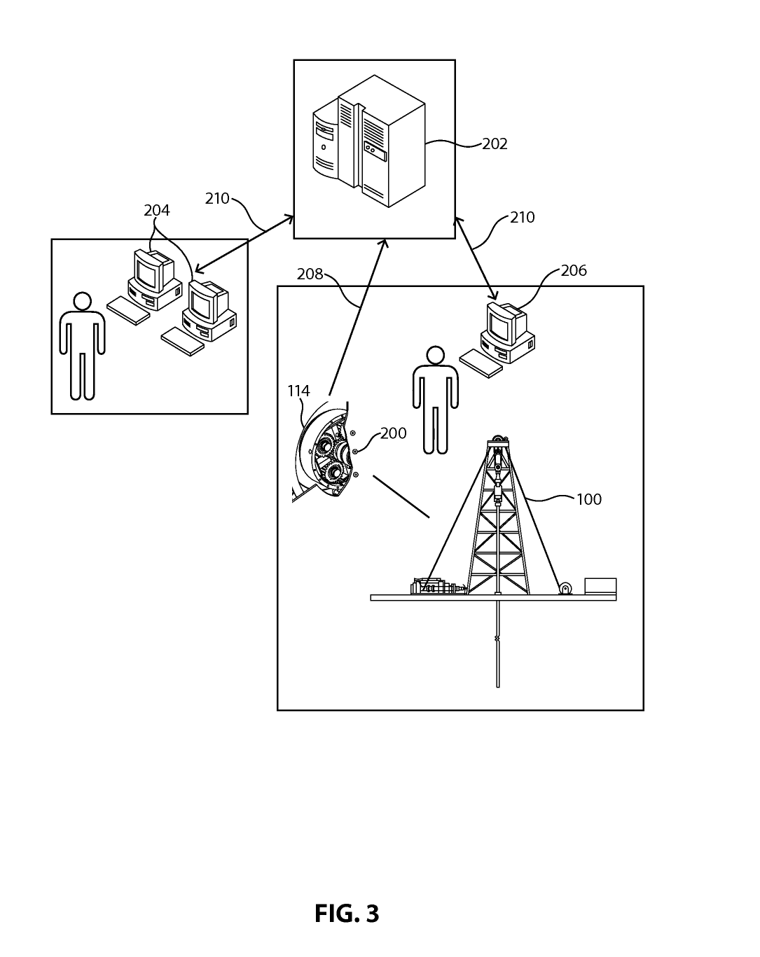

In accordance with an embodiment of the present invention, FIG. 1 is a simplified schematic of a subterranean drilling operation 100. FIG. 3 is a simplified schematic of an associated active sensing and continuous (real time) information relaying operation adapted to sense and actively relay a condition of a heavy weight lubrication oil.

Referring now to FIG. 1, a drilling rig 100 can generally include a substructure 102 and a derrick 104. The derrick 104 can be attached to the substructure 102 and can extend therefrom. The derrick 104 can be a tower or a guyed mast such as a pole which can be hinged at a bottom end. The derrick 104 and substructure 102 can be permanent or can be adapted to break down for transportation.

The drilling rig 100 can be an offshore drilling rig or a land based drilling rig. Offshore drilling rigs can take many forms. For example, the drilling rig 100 can have a fixed platform or substructure attached to an underlying seabed. Alternatively, the drilling rig 100 can include a floating platform disposed at least partially underwater with an anchoring system holding the drilling rig 100 relatively near the underwater drilling operation. It should be understood that the particular configuration and embodiment of the drilling rig 100 are not intended to limit the scope of the present disclosure.

In particular embodiments, the drilling rig 100 can further include a hoisting system 106, a rotating system 108, and a power supply 110. The derrick 104 can support the hoisting system 106 and the rotating system 108. In a particular embodiment, the hoisting system 106 can include a drawworks 114 and a block and tackle system 116 adapted to support a drill string 118.

It should be understood that the particular configuration and embodiment of the drilling rig 100 are not intended to limit the scope of the present disclosure.

In a particular aspect, at least one sensor 200 can be disposed on an equipment of the drilling rig 100 to actively sense and generate data regarding a condition of a heavy weight lubrication oil disposed in the equipment. As used herein, “actively sense” refers to an act of sensing where a sensing condition occurs at least once every hour, such as at least once every 30 minutes, at least once every minute, or even at least once every 10 seconds. In a particular embodiment, “actively sense” refers to an act of sensing wherein a sensing condition occurs at least 1 time per minute (TPM), such as at least 30 TPM, at least 60 TPM, at least 120 TPM, or even at least 300 TPM. Moreover, in particular embodiments, the sensors can sense the condition no greater than 5,000 TPM, such as no greater than 4,000 TPM, no greater than 3,000 TPM, no greater than 1,000 TPM, no greater than 500 TPM, or even no greater than 300 TPM.

In an embodiment, the lubrication oil can be a heavy weight lubrication oil. As used herein, “a heavy weight lubrication oil” refers to a viscous lubricating oil adapted for use in equipment, such as, for example, gears, transmissions, transfer cases, differentials, and other machinery. More particularly, “a heavy weight lubrication oil” can refer to a lubrication oil having a weight, as measured according to the Society of Automotive Engineers (SAE) of at least 75 W, such as at least 80 W, at least 85 W, at least 90 W, at least 140 W, or even at least 250 W. Such heavy weight lubrication oils have a higher viscosity than conventional motor oil and are traditionally utilized in heavy machinery.

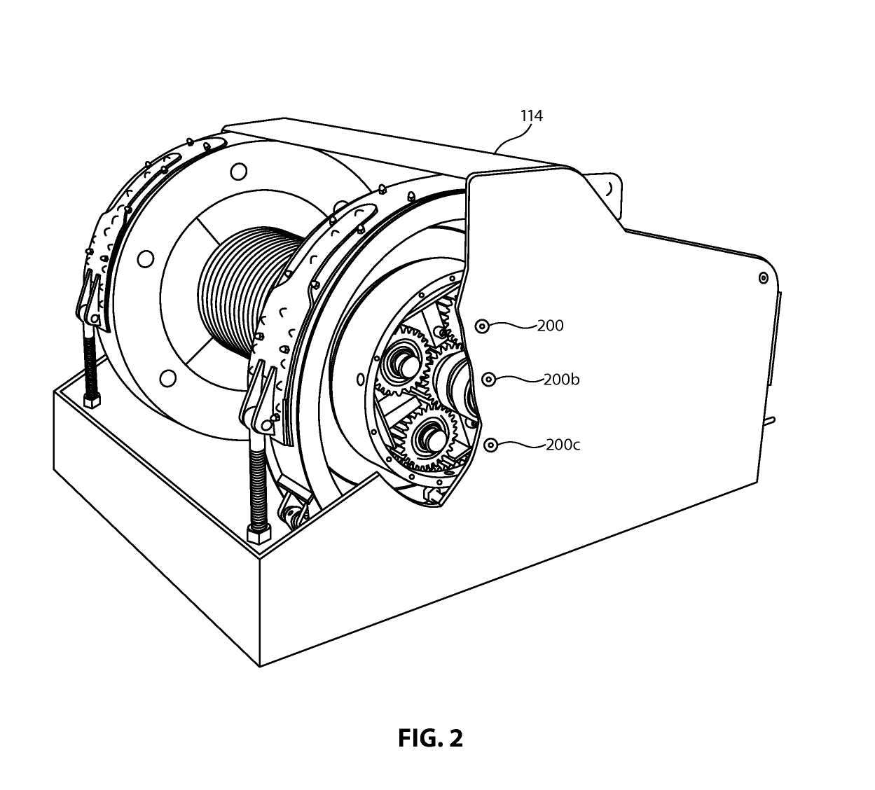

As contemplated herein, the sensors 200 can be disposed on various equipment of the drill rig 100 that require a heavy weight lubrication oil. For example, the sensors 200 can be disposed on the top drive 108, the draw works 114 (FIG. 2), the power supply 110, a transmission associated with any of those equipment, or any other similar equipment. In a certain embodiment, a single sensor 200 can be disposed on each article of equipment. Referring to FIG. 2, in another embodiment, a plurality of sensors 200, 200b, and 200c can be disposed on each article of equipment. Because the heavy weight lubrication oil may have different conditions at different physical locations within the equipment, each of the plurality of sensors 200, 200b, and 200c can be positioned at different locations, e.g., different vertical positions, within each article of equipment. The sensed data from each sensor 200, 200b, and 200c can be individually monitored or, alternatively, can be averaged with the sensed data from all of the sensors to generate an average sensed condition value.

Heavy weight lubrication oil is typically disposed within a housing of articles of equipment where moving members are positioned, e.g., bearings, gears, pistons, or any other moving components. In this regard, the sensors 200 can be at least partially disposed within the housing of the equipment. In certain embodiments, at least a portion of the sensors 200 can be in fluid communication with the heavy weight lubrication oil. In further embodiments, all of each sensor 200 can be disposed within the housing. In this regard, all of each sensor 200 can be in fluid communication with the heavy weight lubrication oil. In another embodiment, all of each sensor 200 can be disposed outside of the housing of the equipment. In such a manner, a fluid return line can extend from the housing and fluidly connect to the sensor. In this regard, the sensors 200 can be disposed outside of the housing.

In a particular embodiment, the sensors 200 can be in communication with an intermediary member disposed outside of the housing by electrical wiring extending through the housing. The electrical wiring can extend through a bore of the housing to an outer surface of the equipment. A sufficiently resilient sealing material can be disposed between the bore of the housing and the electrical wiring in order to seal the bore and prevent leakage therethrough. In another embodiment, the sensors 200 can be in communication with an intermediary member disposed outside of the housing by a wireless signal. In this regard, the sensors 200 can communicate the sensed condition to an intermediary member located outside of the housing of the equipment. In another embodiment, the sensors 200 can be directly engaged with a logic element 202, independent of an intermediary member. The logic element 202 may be disposed immediately proximate to the sensors.

In a non-limiting embodiment, it may be advantageous to position at least a portion of each sensor 200 at a location whereby the sensor 200 can be reached and affected from an exterior location of the equipment. In another non-limiting embodiment, the sensors 200 can be disposed on a portion of the equipment that can be readily removed or opened in order to expose the sensor, e.g., a sealable hatch or access point. In such a manner, the sensors 200 can be manipulated, adjusted, or even replaced without requiring significant operation upon the equipment.

Referring now to FIG. 3, during drilling operations, one or more sensors 200 can actively monitor a condition of the heavy weight lubrication oil. For example, the sensors 200 can monitor a viscosity of the heavy weight lubrication oil, a density of the heavy weight lubrication oil, a dielectric of the heavy weight lubrication oil, a temperature of the heavy weight lubrication oil, or any combination thereof. In a particular embodiment, the sensors 200 can actively monitor all four conditions of the heavy weight lubrication oil simultaneously, or in successive measurements, e.g., alternating between sensed measurements.

After being collected by the sensors 200, a sensed data relating to the condition of the heavy weight lubrication oil can be transferred (illustrated by line 208) continuously (in real time) to a logic element 202. As used herein “transferred continuously” refers to a transmission of data at least once every hour, such as at least once every 30 minutes, at least once every minute, or even at least once every 10 seconds. In a particular embodiment, “transferred continuously” refers to a transmission of data at least once every 30 minutes. In yet a more particular embodiment, “transferred continuously” refers to the transmission of data as it is obtained at each sensed interval, i.e., data is immediately transferred from the sensors to the logic element. In a particular embodiment, a memory storage unit can be attached to the sensors 200 for the temporary storage of the sensed data prior to transfer. The memory storage unit can further include a back up power supply.

In a particular embodiment, the sensed data can be transferred to the logic element 202 as one or more data streams over a network or other wireless signal. For example, in a particular embodiment, a satellite communication element can relay the sensed data through a satellite relay system to a remote geographic location, disposed at a location different than the drill rig. In a particular embodiment, the transfer format and protocol can be based on the industry WITSML format, which uses XML as a data format and web services over HTTPS as a protocol. In another embodiment, the sensed data can be transferred directly to the logic element 202 by wiring or by another non-wireless local communication system, such as a LAN network. In such a manner, the logic element 202 can be disposed at a location on, or proximate to, the drill rig. In yet another embodiment, the logic element can include a plurality of interconnected logic elements. The interconnected logic elements can all be disposed at a single location or at separation locations interconnected by network or wireless signal.

In a particular embodiment, the logic element 202 can include a programmable logic controller, such as a computer software. The logic element 202 can be adapted to receive a signal generated by the sensor 200—the signal containing sensed data regarding the condition of the heavy weight lubrication oil.

Utilizing the data contained in the signal, the logic element 202 can perform a calculation and generate an alarm signal when the sensed condition deviates from an accepted value by more than 5%, such as when the condition deviates from the accepted value by more than 10%, or even when the condition deviates from the accepted value by more than 15%. The alarm signal can indicate to a user or drilling engineer that the condition of the heavy weight lubrication oil contained in the equipment is outside of an acceptable range of the accepted value.

In a particular embodiment, the accepted value can be programmed by a user, i.e., a user can formulate an acceptable value for the measured conditions and set the accepted value accordingly in the logic element. Moreover, the value for the deviation range can be custom selected based on operational factors. In this regard, a user can adjust the deviation calculation based on environmental factors or risk assessment. For example, in harsh climates, e.g., deep water, dessert, or tropical locations, a lower deviation (e.g., 5% from accepted value) can be utilized as the alarm generating condition. In less risk averse drilling operations, e.g., small scale on-land operations, a higher deviation (e.g., 20%) can be utilized as the alarm generating condition. In such a manner, risk can be assessed and addressed on a per operation manner.

In another embodiment, the accepted value can be set by one or more of the previously sensed conditions, e.g., the accepted value can be determined based on a previously sensed value of the condition. For example, the accepted value can be determined by a first value sensed by the sensor, to which all future deviations are measured and compared against. If a later sensed value deviates from the initially allotted value to a degree beyond the allotted deviation, an alarm signal can be generated.

After performing an analysis of the sensed condition, the logic element 202 can communicate (illustrated by lines 210) a signal to an interface 204. The interface 204 can include a user interface adapted to display the signal from the logic element 202. In this regard, a user can visually determine the condition (wear, contamination, etc.) of the heavy weight lubrication oil. In another embodiment, the logic element 202 can transfer the signal to an interface 206 located at the drill site.

In certain embodiments, the interface 204 can display to a user one of two indications—an indication that the sensed condition of the heavy weight lubrication oil is within the acceptable range or an indication that the sensed condition of the heavy weight lubrication oil is outside of the acceptable range. A third indication may optionally indicate to the user that the condition of the heavy weight lubrication oil is approaching the acceptable deviation, i.e., the oil may need to be replaced soon.

In another embodiment, the interface 204 can provide a real-time numerical visualization of the sensed condition of the heavy weight lubrication oil. In this regard, the interface 204 may further include a visualization tool including graphical comparisons through time-indexed graphs. The visualization tool may be capable of illustrating qualitative parameter values, trends, interpreted activities, interesting events, etc. for the purpose of enhancing overall operation.

In the case of a rapid fluctuation of the sensed condition, visualization tool may not be sufficient to rapidly alert of impending heavy weight lubrication oil failure. In this regard, in certain embodiments it may be desirable to include an indicator to indicate whether the condition of the condition of the heavy weight lubrication oil is within or outside of the acceptable range.

The interface 204 can additionally include a data analysis server. Drilling engineers and other users and operators can use a client application running a personal computer or other computing device to connect from the drilling rig site or an operations center to the data analysis server in order to receive and display the sensed data. Once connected, the client application can be continuously updated with information from the data analysis server until such a time as the client is closed. In a particular embodiment, the data analysis server can be a program written in a Java programming language. The preferred client application can also be a Java application. The protocol between the client application and the server application can be based on regular polling by the client application using an HTTP or HTTPS (secured) connection.

A memory element can be positioned to interact with one or more of the logic element, the interface, or the data analysis server, and record and store historical valuation calculations for future analysis and review. The memory element can be disposed at a location proximate to the drill rig, the logic element, the interface, the data analysis server, or any other suitable location. The memory element can optionally contain a programmable software adapted to erase stored recorded data after a threshold period, e.g., every six months, in order to reduce required storage capacity.

In a certain embodiment, the system for wellbore operations can further include a stop element adapted to permit a user to terminate drilling operations in the case of an emergency. The stop element can be handled by an operator located at the interface. In this regard, any active drilling operations can be shut down remotely and a service crew can be dispatched to the drill site.

In a particular embodiment, the system for wellbore operations may further include a sampling feature adapted to siphon, remove, and store a sample of the heavy weight lubrication oil. The sampling feature can allow a user to retroactively analyze a sample of the heavy weight lubrication oil and better formulate tolerances and deviations for future operations.

Many different aspects and embodiments are possible. Some of those aspects and embodiments are described below. After reading this specification, skilled artisans will appreciate that those aspects and embodiments are illustrative and do not limit the scope of the present invention. Embodiments may be in accordance with any one or more of the items as listed below.

Item 1. A system for wellbore operations adapted to generate an alarm signal upon ingress of a contaminant into a heavy weight lubrication oil of an equipment prior to operational failure of the equipment.

Item 2. A system for wellbore operations comprising a drill rig having an equipment with a heavy weight lubrication oil, and a sensor disposed on the equipment, wherein the sensor is adapted to sense for ingress of a contaminant into the heavy weight lubrication oil.

Item 3. A system for wellbore operations comprising:
an equipment having a heavy weight lubrication oil;
a sensor disposed on the equipment, the sensor adapted to sense a condition of the heavy weight lubrication oil; and
a logic element in communication with the sensor, the logic element adapted to receive a signal from the sensor containing an information regarding the condition of the heavy weight lubrication oil.

Item 4. A system for wellbore operations comprising:
an equipment, the equipment containing a heavy weight lubrication oil;
a sensor at disposed on the equipment, the sensor adapted to sense a condition of the heavy weight lubrication oil, the condition including:
   a viscosity of the heavy weight lubrication oil;
   a density of the heavy weight lubrication oil;
   a dielectric of the heavy weight lubrication oil;
   a temperature of the heavy weight lubrication oil; or
   a combination thereof; and
a logic element in communication with the sensor, the logic element adapted to receive a signal from the sensor containing an information regarding the condition of the heavy weight lubrication oil.

Item 5. The system according to item 1, further comprising a sensor at least partially disposed within the equipment, the sensor adapted to sense a condition of a heavy weight lubrication oil contained in the equipment.

Item 6. The system according to any one of items 2-5, wherein the sensor is fully disposed within the equipment.

Item 7. The system according to any one of items 2, 3, 5, or 6, wherein the sensor is adapted to sense:
a viscosity of the heavy weight lubrication oil;
a density of the heavy weight lubrication oil;
a dielectric of the heavy weight lubrication oil;
a temperature of the heavy weight lubrication oil; or
a combination thereof.

Item 8. The system according to any one of items 3-7, wherein the sensor is adapted to actively sense the condition of the heavy weight lubrication oil.

Item 9. The system according to any one of items 3-8, wherein the sensor is adapted to sense the condition of the heavy weight lubrication oil at least 1 time per minute (TPM), such as at least 30 TPM, at least 60 TPM, at least 120 TPM, or even at least 300 TPM.

Item 10. The system according to item 9, wherein the sensor is adapted to sense the condition of the heavy weight lubrication oil no greater than 5,000 TPM, such as no greater than 4,000 TPM, or no greater than 3,000 TPM.

Item 11. The system according to any one of items 2-10, wherein the sensor is adapted to generate a signal regarding the condition of a heavy weight lubrication oil in the equipment.

Item 12. The system according to any one of items 3 or 4, wherein the condition of the heavy weight lubrication oil is affected by ingress of a contaminant into the equipment.

Item 13. The system according to any one of items 1, 2, or 12, wherein the contaminant comprises a mud.

Item 14. The system according to any one of items 2 or 5-13, further comprising a logic element in communication with the sensor, the logic element adapted to receive a signal from the sensor.

Item 15. The system according to any one of items 3, 4, or 14, wherein the logic element comprises a programmable logic controller.

Item 16. The system according to any one of items 3, 4, 14, or 15, wherein the logic element comprises a software adapted to generate an alarm signal upon ingress of a contaminant into the equipment.

Item 17. The system according to item 16, wherein the software is preprogrammed into the logic element.

Item 18. The system according to any one of items 16 or 17, wherein the software is programmable to receive an accepted value, and wherein the software is adapted to analyze the signal from the sensor and compare the signal against the accepted value.

Item 19. The system according to item 18, wherein the software is adapted to generate an alarm signal in response to analyzing the signal from the sensor.

Item 20. The system according to any one of items 3, 4, or 14-19, wherein the logic element is adapted to generate an alarm signal when the condition as transmitted from the signal of the sensor deviates from an accepted value by more than 5%, such as when the condition deviates by more than 10%, or even when the condition deviates by more than 15%.

Item 21. The system according to any one of items 3, 4, 14-20, wherein the logic element is disposed at a location proximate to the wellbore.

Item 22. The system according to any one of items 3, 4, 14-20, wherein the logic element is disposed at a location geographically removed from the wellbore.

Item 23. The system according to any one of items 3, 4, 14-22, wherein the logic element comprises a plurality of logic elements.

Item 24. The system according to item 23, wherein each logic element of the plurality of logic elements is disposed at a single geographic location.

Item 25. The system according to item 23, wherein each logic element of the plurality of logic elements is disposed at a separate geographic location.

Item 26. The system according to any one of items 24 or 25, wherein at least two logic elements of the plurality of logic elements are in communication.

Item 27. The system according to any one of items 24-26, wherein all logic elements of the plurality of logic elements are in communication.

Item 28. The system according to any one of items 3, 4, or 14-27, further comprising a first interface in communication with the logic element.

Item 29. The system according to item 28, wherein the first interface comprises a user interface.

Item 30. The system according to any one of items 28 or 29, wherein the first interface comprises a monitor adapted to display an alarm signal generated by the logic element to a user.

Item 31. The system according to any one of items 28-30, wherein the first interface is disposed at a location proximate to the wellbore.

Item 32. The system according to any one of items 28-30, wherein the first interface is disposed at a location geographically removed from the wellbore.

Item 33. The system according to any one of items 28-32, further comprising a second interface, the second interface disposed at a location geographically removed from a location of the first interface.

Item 34. The system according to item 33, wherein the second interface comprises a user interface.

Item 35. The system according to item 33, wherein the second interface comprises a server.

Item 36. The system according to item 33, wherein the second interface comprises a network adapted to be accessible by remote access.

Item 37. A method of monitoring a condition of an equipment on a drill rig comprising:
sensing a condition of a heavy weight lubrication oil disposed within an equipment, the condition including a viscosity, a density, a dielectric, a temperature, or any combination thereof of the heavy weight lubrication oil; and
transmitting the condition to a logic element.

Item 38. The method according to item 37, wherein sensing is performed by a sensor disposed at least partially within the equipment.

Item 39. The method according to any one of items 37 or 38, wherein sensing is performed such that a sensor senses:
a viscosity of the heavy weight lubrication oil;
a density of the heavy weight lubrication oil;
a dielectric of the heavy weight lubrication oil;
a temperature of the heavy weight lubrication oil; or
a combination thereof.

Item 40. The method according to any one of items 37-39, further comprising:
analyzing the condition.

Item 41. The method according to any one of items 37-40, further comprising:
generating an alarm signal when the condition deviates from an accepted value by more than 5%, such as when the condition deviates by more than 10%, or even when the condition deviates by more than 15%.

Item 42. The method according to any one of items 37-41, further comprising:
automatically adjusting a fluid characteristic of the heavy weight lubrication oil upon a threshold condition.

Item 43. The method according to item 42, wherein automatically adjusting a fluid characteristic comprises:
stopping operation of the equipment.

Item 44. The method according to any one of items 42 or 43, wherein automatically adjusting a fluid characteristic comprises:
draining the heavy weight lubrication oil from the equipment.

Item 45. The method according to any one of items 42-44, wherein automatically adjusting a fluid characteristic comprises:
filling a new heavy weight lubrication oil into the equipment.

Item 46. The method according to any one of items 42-45, wherein automatically adjusting a fluid characteristic comprises:
restarting operation of the equipment.

Item 47. The system or method according to any one of the preceding items, wherein the heavy weight lubrication oil has at least a 90 weight, such as at least a 140 weight, at least a 200 weight, or even at least a 250 weight, as measured according to the Society of Automotive Engineers.

Item 48. A system for wellbore operations comprising a logic element adapted to receive a signal containing information regarding a viscosity, density, dielectric, and temperature of a heavy weight lubrication oil within an equipment and generate an alarm signal when the condition deviates from an accepted value by more than 5%, such as when the condition deviates by more than 10%, or even when the condition deviates by more than 15%.

Item 49. A system for wellbore operations comprising an interface adapted to alert a user when a condition of a heavy weight lubrication oil in an equipment deviates from an accepted value by more than 5%, such as when the condition deviates by more than 10%, or even when the condition deviates by more than 15%.

Item 50. The system according to any one of items 48 and 49, wherein the logic element comprises a programmable logic controller.

Item 51. The system according to any one of items 48-50, wherein the logic element comprises a software.

Item 52. The system according to item 51, wherein the software comprises at least one predefined condition, and wherein the software is adapted to analyze the condition as send in the signal from the sensor.

Item 53. The system according to item 52, wherein the software is programmable to receive an accepted value, and wherein the software is adapted to analyze the signal from the sensor and compare the data contained in the signal against the accepted value.

Item 54. The system according to any one of items 51 or 52, wherein the software is adapted to generate an alarm signal upon a calculated deviation between the condition and the predefined condition by more than 5%, such as when the condition deviates from the predefined condition by more than 10%, or even when the condition deviates from the predefined condition by more than 15%.

Item 55. The system according to any one of items 48-54, wherein at least a portion of the logic element is disposed at a location proximate to the wellbore.

Item 56. The system according to any one of items 48-55, wherein at a least a portion of the logic element is disposed at a location geographically removed from the wellbore.

Item 57. The system according to any one of items 48-56, wherein the logic element comprises a plurality of logic elements.

Item 58. The system according to item 57, wherein at least two of the plurality of logic elements are connected.

Item 59. The system according to any one of items 57 or 58, wherein all of the logic elements are interconnected.

Item 60. The system according to any one of items 48-59, wherein the logic element is adapted to communicate with a first interface.

Item 61. The system according to item 60, wherein the first interface comprises a user interface.

Item 62. The system according to any one of items 60 or 61, wherein the first interface is disposed at a different location than the logic element.

Item 63. The system according to any one of items 60-62, wherein the interface is adapted to communicate with a second interface.

Item 64. The system according to item 63, wherein the second interface comprises a user interface.

Item 65. The system according to item 63, wherein the second interface comprises a server.

Item 66. The system according to item 63, wherein the second interface comprises a network adapted to be accessible by remote access.

Item 67. The system according to any one of items 60-66, further comprising a wireless communication system, the wireless communication system adapted to relay the alarm signal from the logic element to the user interface.

Item 68. The system according to item 67, wherein the wireless communication system comprises a satellite transmitter.

Item 69. The system according to any one of items 48-68, wherein the condition comprises a viscosity of the heavy weight lubrication oil; a density of the heavy weight lubrication oil; a dielectric of the heavy weight lubrication oil; and a temperature of the heavy weight lubrication oil.

Item 70. A method of monitoring a condition of an equipment on a drill rig comprising:
receiving, by one or more logic devices, a signal from a sensor, wherein the sensor is disposed in an equipment disposed at a drill rig, and wherein the signal includes a sensed condition of a heavy weight lubrication oil in the equipment;
analyzing the sensed condition;
generating an alarm signal upon ingress of a contaminant into the heavy weight lubrication oil of the equipment.

Item 71. A method of monitoring a condition of an equipment on a drill rig comprising:
receiving, by one or more logic devices, a signal from a sensor, wherein the sensor is disposed in n equipment disposed at a drill rig, and wherein the signal includes a sensed condition of a heavy weight lubrication oil in the equipment;
analyzing the sensed condition;
generating an alarm signal when the condition deviates from an accepted value by more than 5%, such as when the condition deviates by more than 10%, or even when the condition deviates by more than 15%.

Item 72. The method according to any one of items 70 or 71, further comprising: the logic element generating a pass signal when the condition is within an acceptable range.

Item 73. The method according to any one of items 70-72, further comprising:
transmitting to an interface the alarm signal or the pass signal.

Item 74. The method according to item 73, wherein transmitting the alarm signal is performed by a wireless signal.

Item 75. The method according to any one of items 70-74, wherein the sensor is adapted to sense:
a viscosity of the heavy weight lubrication oil;
a density of the heavy weight lubrication oil;
a dielectric of the heavy weight lubrication oil;
a temperature of the heavy weight lubrication oil; or
a combination thereof.

Item 76. The method according to any one of items 70-75, wherein the sensor is adapted to actively sense the condition.

Item 77. The method according to any one of items 70-76, wherein the sensor is adapted to sense the condition of the heavy weight lubrication oil at least 1 time per minute (TPM), such as at least 30 TPM, at least 60 TPM, at least 120 TPM, or even at least 300 TPM.

Item 78. The method according to any one of items 70-77, wherein the sensor is adapted to transmit the condition to the logic element upon each discrete sensing interval.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

The embodiments provide a combination of features, which can be combined in various matters to describe and define a method and system of the embodiments. The description is not intended to set forth a hierarchy of features, but different features that can be combined in one or more manners to define the invention. In the foregoing, reference to specific embodiments and the connection of certain components is illustrative. It will be appreciated that reference to components as being coupled or connected is intended to disclose either direct connected between said components or indirect connection through one or more intervening components as will be appreciated to carry out the methods as discussed herein.

As such, the above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

The disclosure is submitted with the understanding that it will not be used to interpret or limit the scope or meaning of the claims. In addition, in the foregoing disclosure, various features may be grouped together or described in a single embodiment for the purpose of streamlining the disclosure. This disclosure is not to be interpreted as reflecting an intention that the embodiments herein limit the features provided in the claims, and moreover, any of the features described herein can be combined together to describe the inventive subject matter. Still, inventive subject matter may be directed to less than all features of any of the disclosed embodiments.

What is claimed is:

1. A system for wellbore operations comprising:
an equipment having a heavy weight lubrication oil;
a sensor disposed on the equipment, the sensor adapted to sense a condition of the heavy weight lubrication oil; and
a logic element in communication with the sensor, the logic element adapted to receive a signal from the sensor containing an information regarding the condition of the heavy weight lubrication oil.

2. The system according to claim 1, wherein the sensor is fully disposed within the equipment.

3. The system according to claim 1, wherein the sensor is adapted to sense:
a viscosity of the heavy weight lubrication oil;
a density of the heavy weight lubrication oil;
a dielectric of the heavy weight lubrication oil;
a temperature of the heavy weight lubrication oil; or
a combination thereof.

4. The system according to claim 1, wherein the sensor is adapted to actively sense the condition of the heavy weight lubrication oil.

5. The system according to claim 1, wherein the sensor is adapted to sense the condition of the heavy weight lubrication oil at least 1 time per minute.

6. The system according to claim 1, wherein the logic element comprises a software adapted to generate an alarm signal upon ingress of a contaminant into the equipment.

7. The system according to claim 6, wherein the software is programmable to receive an accepted value, and wherein the software is adapted to compare the signal against the accepted value.

8. The system according to claim 7, wherein the software is adapted to generate an alarm signal in response to analyzing the signal from the sensor.

9. The system according to claim 1, wherein the logic element is adapted to generate an alarm signal when the condition as transmitted from the signal of the sensor deviates from an accepted value by more than 15%.

10. The system according to claim 1, wherein the logic element is disposed at a location geographically removed from the wellbore.

11. The system according to claim 1, further comprising a first interface in communication with the logic element.

12. The system according to claim 11, wherein the first interface comprises a user interface.

13. The system according to claim 11, wherein the first interface is disposed at a location geographically removed from the wellbore.

14. The system according to claim 11, further comprising a second interface disposed at a location geographically removed from the first interface.

15. The system according to claim 14, wherein the second interface comprises a network adapted to be accessible by remote access.

16. The system according to claim 1, further comprising a wireless communication system, the wireless communication system adapted to relay data from the logic element to a user interface.

17. A method of monitoring a condition of an equipment on a drill rig comprising:
sensing a condition of a heavy weight lubrication oil disposed within an equipment, the condition including a viscosity, a density, a dielectric, a temperature, or any combination thereof of the heavy weight lubrication oil; and
transmitting the condition to a logic element.

18. The method according to claim 17, further comprising:
generating an alarm signal when the condition deviates from an accepted value by more than 15%.

19. The method according to claim 17, further comprising:
automatically adjusting a fluid characteristic of the heavy weight lubrication oil upon a threshold condition.

20. A system for wellbore operations comprising an interface adapted to alert a user when a condition of a heavy weight lubrication oil in an equipment on a drill rig deviates from an accepted value by more than 15%.
